(19) United States
(12) Patent Application Publication
(10) Pub. No.: US 2016/0095593 A1
(43) Pub. Date: Apr. 7, 2016

(54) STAPLING DEVICE WITH LOCKING MECHANISM

(71) Applicant: Airbus Operations GmbH, Hamburg (DE)

(72) Inventors: Dirk Schumacher, Hamburg (DE); Hamid Ebrahimi, Scharnebeck (DE)

(73) Assignee: Airbus Operations GmbH, Hamburg (DE)

(21) Appl. No.: 14/867,486

(22) Filed: Sep. 28, 2015

(30) Foreign Application Priority Data
Oct. 1, 2014 (DE) 10 2014 114 300.0

(51) Int. Cl. A61B 17/068; A61B 17/064

(52) U.S. Cl. CPC A61B 17/0684; A61B 17/0644; A61B 2017/0647

(57) ABSTRACT

A stapling device includes a stapling needle system that is guided through the base body of the stapling device. The stapling needle system can be passed through a hole in one or more components. By exerting a tensile force on the stapling needle system, the component or components can be sandwiched between the stapling needle head and base body of the stapling device. The stapling needle system can be positively locked with the base body by means of a locking device, so as to lock the stapling device in place.

### FIELD OF THE INVENTION

The present invention relates to the temporary clamping of components. In particular, the present invention relates to a stapling device and a stapling system.

### BACKGROUND OF THE INVENTION

So-called stapling screwdrivers can be used to temporarily join components together before they are ultimately fixed with other components. These usually encompass a base body and stapling needle, wherein the components to be clamped can be sandwiched between a stapling needle head of the stapling needle and the base body. To this end, the stapling needle can usually be guided through a hole in the component, so that the components can then be sandwiched between the stapling needle head and base body.

In order to achieve the clamping, the stapling needle or stapling needle head can be retracted by means of a threaded rod via a screwing movement, wherein the stapling needle head spreads apart while retracting the stapling needle, so that the stapling needle head jams with the hole in the components, and the clamping of components can be achieved.

The threaded rod can here be tightened with a screw thread. This tightening of the threaded rod via a screwing movement can be relatively time-consuming and require special tools.

### BRIEF SUMMARY OF THE INVENTION

A first aspect of the present invention relates to a stapling device for temporarily clamping a component, wherein the stapling device comprises a stapling needle system exhibiting a stapling needle and stapling needle head, as well as a base body exhibiting an interior space for guiding the stapling needle system. The stapling device further comprises a locking mechanism for locking the stapling needle system relative to the base body with a positive fit. The stapling needle system can be displaced relative to the base body by applying a tensile force to the stapling needle system in the direction of a longitudinal axis of the stapling needle system. The stapling device is further designed to clamp the component between the stapling needle head and base body.

This may provide an easy to operate device for temporarily joining together components.

In other words, the stapling device may be understood as a device for temporarily joining together components that comprises a stapling needle system, which is guided through the base body of the stapling device. The stapling needle system may be guided through a hole in one or more components. By exerting a tensile stress on the stapling needle system, the component or components can be sandwiched between the stapling needle head and base body of the stapling device.

The device may also be designed for temporarily clamping a plurality of components.

The stapling needle system may comprise several elements, which can be rigidly joined together. The stapling needle can be designed to be guided through a hole in the component. For example, the stapling needle system may comprise a stapling needle and stapling needle axis that are rigidly joined together. The stapling needle of the stapling needle system may be formed as a single piece and/or can be made out of a forged material. The stapling needle head may denote an area of the stapling needle that can be spread apart radially to the longitudinal direction of the stapling needle. In other words, the diameter of the stapling needle head may be variable. The stapling needle may be machined in such a way that at least the stapling needle head is flexible. In other words, the process of spreading apart the stapling needle head can be reversible, so that the diameter of the stapling needle head diminishes again once the stapling needle is detached. In this way, the stapling needle may be removed from the hole in the components. For example, the stapling needle head may be situated at an end of the stapling needle, preferably at the end remote from the base body of the stapling device. Viewed from the top down, for example, the stapling needle system may exhibit a tensile head, the stapling needle axis, the stapling needle and the stapling needle head.

For example, the stapling needle may have a length of 114 mm to 130 mm, and a diameter of 4 mm. The length of the stapling needle may further depend on the packet thickness of the components to be stapled, wherein the packet thickness may measure between 4 mm and 20 mm, for example. For example, the stapling needle head diameter may measure 15 mm, and the housing diameter may measure 18 mm.

The base body may be understood as a component or element of the stapling device that is designed to accommodate and/or guide the stapling needle system.

For example, the base body may be made out of metal. The interior space of the base body can exhibit one or more holes. For example, the cross section of the interior space of the base body can be adjusted to the stapling needle system in such a way as to enable only a translational motion by the stapling needle system relative to the base body along one direction.

The stapling needle system may comprise a longitudinal axis that is parallel to the longitudinal axis of the stapling needle. By applying a tensile force or compressive force to the stapling needle system, the stapling needle system can be moved relative to the base body in the interior space of the base body. In other words, no screwing movement of the stapling needle system relative to the base body is required to cause the stapling needle system to move in the direction of the longitudinal axis of the stapling needle system relative to the base body.

The locking mechanism may be used to positively lock or bolt the stapling needle system with the base body. A positive connection can be regarded as the intermeshing of at least two joining partners, e.g., a tooth element and a tooth system. Put differently, one joining partner can block the other one, and thereby prevent it from moving in a positive connection. In other words, the stapling needle system can be locked to clamp the components, so that the locking mechanism prevents a relative movement between the stapling needle system and base body to release the clamped components. This means that the position of the stapling needle system can be fixed relative to the base body. Positive locking may here be understood to mean that a first element the base body, e.g., a tooth element, is positively joined with a second element secured to the stapling needle system, e.g., a tooth system, to prevent a relative movement between the stapling needle system and base body. This lock may also be reversed again so as to detach the stapling device from the component or components once more. For example, the positive connection between the first element, e.g., the tooth element, and the second element, e.g., the tooth system, may be undone to release the lock.

The stapling device can be used in the structural assembly of a hull in aircraft construction or shipbuilding. For example, the stapling device can be used for sealing longitudinal and transverse seams. The stapling device can further be used in various areas of industrialized building requiring that several parts be temporarily held together.

The stapling device according to the invention may offer the advantage of being easy to mount and clamp from one side of the component. A screwing movement of the stapling needle system is not required for clamping the stapling device, so that the stapling device can be clamped with a simple movement. In addition, no cost-intensive and/or heavy tools must be provided for clamping the stapling device. Furthermore, the stapling device offers the advantage of simultaneously being able to easily achieve a high clamping force and simple release of the stapling device. For example, the stapling device can be clamped with tongs of the kind described within the context of the invention, and then released again with a simple movement, without a tool.

In an exemplary embodiment of the invention, the locking mechanism comprises a tooth system situated on the stapling needle system and a tooth element situated on the base body. The tooth element situated on the base body for positively locking the stapling needle system is further designed to engage in the tooth system situated on the stapling needle system.

In the context of the invention, the tooth system may be understood as a toothed rod arranged on the stapling needle system or integrated into the stapling needle system. The tooth system can exhibit an oblong expansion, wherein the longitudinal direction of the tooth system is parallel to a longitudinal axis of the stapling needle system. The tooth element can exhibit a single tooth or several teeth, which is/are designed in such a way as to engage into the tooth system of the stapling needle system. The tooth element can be secured to the base body or integrated into the base body. In this way, the tooth element can latch into and/or positively lock into various positions in the tooth system of the stapling needle system, making it possible to clamp components of different thicknesses. The locking mechanism can also be referred to as a ratchet system.

In an exemplary embodiment of the invention, the locking mechanism further comprises a locking lever pivoted to the base body. The locking lever exhibits the tooth element, wherein the stapling device exhibits a force element for exerting a force on the locking lever, which presses the tooth element in the direction of the tooth system situated on the stapling needle system.

The locking lever can be pivoted to the base body on a fulcrum or an axis. The lever can further be pivoted from a locked state of the stapling device, in which the tooth element positively engages into the tooth system, into a non-locked state or released state, in which the tooth element does not engage into the tooth system. A user can pivot the locking lever like this in a simple movement, for example, so that the stapling device can be easily detached. For example, the tooth element can protrude out of one end of the locking lever, wherein the locking lever and tooth element can be designed as a single piece. The force element can be a spring, for example, which causes the tooth element to automatically engage into the tooth system, thereby bringing about the positive lock. In order to get from one locking stage to a next locking stage in the locking mechanism, it may be necessary to surmount a specific distance. If the stapling needle system is to be moved by a distance smaller than the distance required to get from one locking stage to the next in order to clamp the components, the locking mechanism can arrive at the next locking stage by additionally compressing a force transmission element of the stapling device described within the context of the invention. The force transmission element can here be designed to be compressed by the distance required to get from one locking stage to the next.

In another exemplary embodiment of the invention, the locking lever exhibits a first arm, a second arm and a fulcrum situated between the first arm and second arm. The locking lever is pivoted to the base body with the fulcrum, wherein the tooth element is situated on the first arm, and the second arm can be actuated to release the stapling needle system.

For example, the second arm can be situated at least partially outside of the base body, so it can be easily grabbed or actuated by the user. The first arm on which the tooth element is situated can be passed through a recess in the base body.

In an exemplary embodiment of the invention, the force element is a tension spring situated around the first arm and around the base body, or a hose spring washer.

In other words, the tension spring or hose spring washer can loop around the first arm as well as the base body. In this way, a high enough force can be exerted to press the tooth element into the tooth system.

In an exemplary embodiment of the invention, the locking mechanism exhibits two locking levers.

For example, the two locking levers can be secured to the base body opposite each other, so that the stapling needle system is situated between the two locking levers. Both locking levers can further each exhibit a tooth element, so that the two tooth elements can engage on two opposite tooth systems of the stapling needle system. In this way, the force for releasing the stapling device can be distributed to two locking levers. For example, the user can detach the stapling device with a thumb and index finger. Both locking levers can each be designed as described in the context of the invention.

In another exemplary embodiment of the invention, the stapling needle device exhibits a flexible force transmission element for transmitting a clamping force to the component, wherein the force transmission element is secured to the base body in such a way that, while the components are being sandwiched between the base body and stapling needle head, the force transmission element is situated between the base body and component, so that the clamping force can be transmitted from the base body to the force transmission element, and from the force transmission element to the components.

In other words, the flexible force transmission element can be understood as a kind of buffer element that can be situated between the component and base body when clamping the component between the stapling needle head and base body. The flexible force transmission element can further be flexible in the direction of the clamping force to be exerted. This means that the flexible force transmission element can absorb the clamping force while clamping the component or components if the clamping force exceeds a specific maximum force to be exerted on the components. For example, the flexible force transmission element can be a spring element, an element comprised of vulcanized or natural rubber, or an element made up of several flexible materials.

Depending on the spring element or plate spring size and plate spring class, the clamping force can vary between 800 N and 2000 N. The maximum spring deflection of the flexible force transmission element or plate spring packet may measure 2 mm. The spring deflection can here depend on the arrangement and/or size of the spring element.

Various distances may be realized as a result. In other words, different spring elements can be used to cover various component thicknesses.

In this way, the clamped components may be protected by the force transmission element. As a consequence, damage to the components can be prevented when clamping the components with the stapling device.

In another exemplary embodiment of the invention, the maximum clamping force that can be transmitted to the component is limited by having the force transmission element transmit the clamping force to the component.

The maximum clamping force that can be transmitted to the component can here be configured by specifically selecting a force transmission element with a specific characteristic or material property. This prevents the stapling device user from being able to exert too high a clamping force on the components, which could damage the components.

In another exemplary embodiment of the invention, the flexible force transmission element is a spring element.

For example, the spring element may be a spiral spring or plate spring packet. In this way, the maximum clamping force can be defined by selecting a spring element with a specific spring constant. In addition, the maximum clamping force of the stapling device can also be varied by changing out the spring element. For example, depending on the component, different stapling devices with various spring elements could be provided, so that a specific clamping force is not exceeded for each component. For example, a stapling device with a diminished clamping force can be provided for clamping components with carbon fiber composite materials.

In another exemplary embodiment of the invention, the stapling device exhibits a thrust bushing for exerting the clamping force on the components. In addition, the force transmission element is situated in the interior space of the base body, and the thrust bushing is situated on the force transmission element in such a way that, while clamping the components, the clamping force can be transmitted from the force transmission element to the thrust bushing, and from the thrust bushing to the components.

In other words, the thrust bushing can be situated between the force transmission element and component. Placing the force transmission element in the interior of the base body makes it possible to situate the force transmission element so as not to be directly accessible from outside. In this way, the stapling device can be configured in such a way that the maximum clamping force transmittable to the component cannot be manipulated by a stapling device user. This makes it possible to increase the safety of the stapling device with respect to potential material damage, since the maximum exertable clamping force cannot be circumvented.

Another aspect of the invention relates to a stapling system that comprises a stapling device described in the context of the present invention along with stapling tongs, wherein the stapling tongs exhibit two expansion jaws for transmitting the clamping force to the base body and stapling needle system, which leads to a displacement of the stapling needle system relative to the base body.

For example, the stapling tongs may be stapling tongs configured in such a way that the expansion jaws of the stapling tongs spread apart when pressing together the tong levers. For example, this can be achieved by having the tongs exhibit a construction with several hinges.

In this way, a simple, relatively cost-effective tool may be provided, which can be simultaneously used for clamping the stapling device and locking the stapling device in a single operational sequence.

The described embodiments relate equally to a stapling device and a stapling system, even though individual embodiments are described exclusively with reference to a stapling device or a stapling system. Synergistic effects may arise from various embodiment combinations, even if they are not described below.

Additional features, advantages and possible applications of the invention may be gleaned from the following description of exemplary embodiments and figures. All described and/or graphically depicted features here comprise the subject matter of the invention, taken in isolation and in any combination, even independently of their composition in the individual claims or back references thereto.

Exemplary embodiments of the present invention will be described below with reference to the figures.

### BRIEF DESCRIPTION OF THE DRAWINGS

FIGS. 1A, 1B and 1C show different sections of a stapling device according to an exemplary embodiment of the invention.

FIGS. 2A and 2B each show a stapling device according to an exemplary embodiment of the invention.

FIGS. 3A and 3B show the process of clamping a component with a stapling device according to an exemplary embodiment of the invention.

FIGS. 4A and 4B show the process of clamping a component with a stapling system according to an exemplary embodiment of the invention.

The figures may be schematic and not shown to scale. If the same reference numbers are indicated in different figures in the description below, they denote identical or similar elements. However, identical or similar elements can also be marked with different reference numbers.

### DETAILED DESCRIPTION

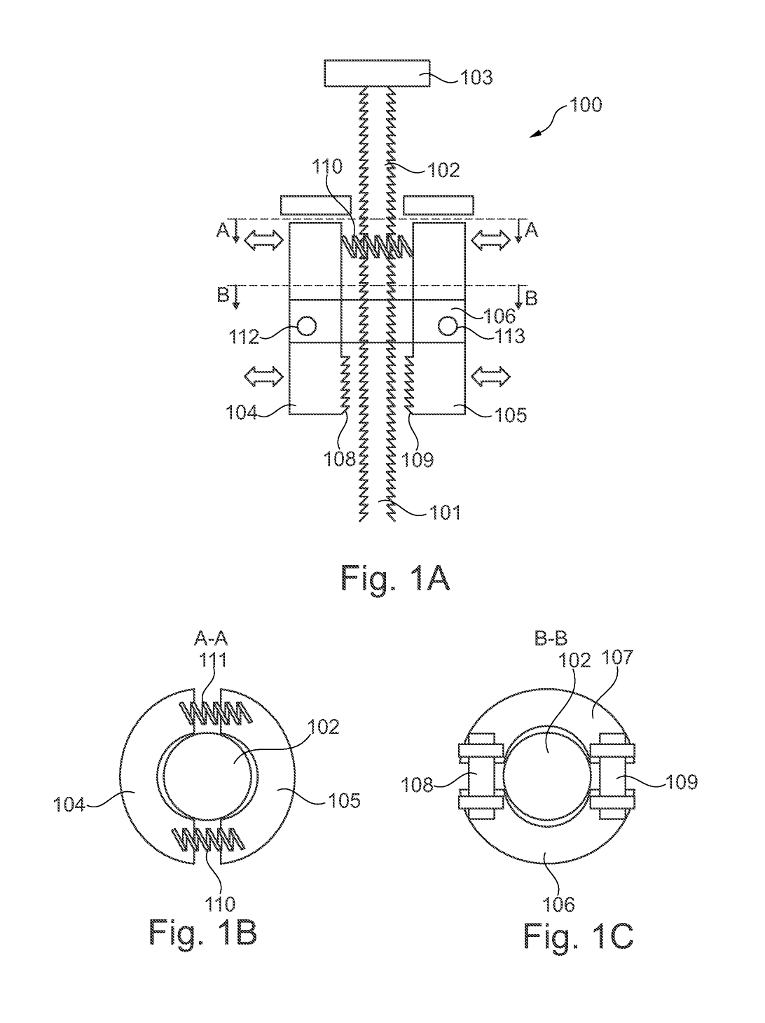

FIG. 1A presents a side view of a stapling device 100 according to an exemplary embodiment of the invention. The stapling device comprises a stapling needle system 101 with a stapling needle axis 102 and a threaded ring or tension head 103 situated at the end of the stapling needle system. The stapling needle system 101 is guided through an interior space of a base body of the stapling device 100, wherein the base body exhibits two opposing base body halves 104 and 105, which are pivoted with a retaining ring exhibiting two retaining ring halves 106 and 107. This means that the first base body half 104 is pivoted to the two retaining ring halves 106 and 107 by way of a first fulcrum 112, and the second base body half 105 is pivoted to the two retaining ring halves 106 and 107 by way of a second fulcrum 113. Situated at the lower end of the two base body halves 104 and 105 are a respective tooth element 108 and 109 on the respective interior side of the base body half 104 and 105, so that the latter can positively engage into a tooth system situated on the stapling needle axis. Also secured between the two base body halves 104 and 105 are two force elements or counter-pressure spiral springs 110 and 111, which exert an outwardly directed force in the upper areas of the base body halves 104 and 105, so that the tooth elements 108 and 109 situated on the lower areas of the base body halves 104 and 105 are pressed into the tooth system of the stapling needle system, thereby automatically latching or locking the stapling device 100. The tooth elements 108 and 109 are designed as a single piece with the base body halves 104 and 105.

In this exemplary embodiment, the stapling needle system 101 is not completely shown. This means that a stapling needle with a stapling needle head is situated under the stapling needle axis 102.

In order to clamp one or more components, the stapling needle is guided through a hole in the component(s), after which the stapling needle system 101 is retracted relative to the base body 104, 105, spreading apart the stapling needle head, thereby causing the component(s) to become clamped between the stapling needle head and lower ends of the base body halves 104 and 105. The force element or counter-pressure spiral spring 110 prompts the tooth elements 108 and 109 to automatically engage into the tooth system of the stapling needle axis, thereby positively locking the clamped stapling device 100. In order to release the stapling device again, the two base body halves 104 and 105 can be pressed together in the upper area, thereby undoing the positive engagement of the tooth elements 108 and 109 in the tooth system of the stapling needle axis 102.

FIGS. 1B and 1C show the sections A-A and B-B depicted on FIG. 1A.

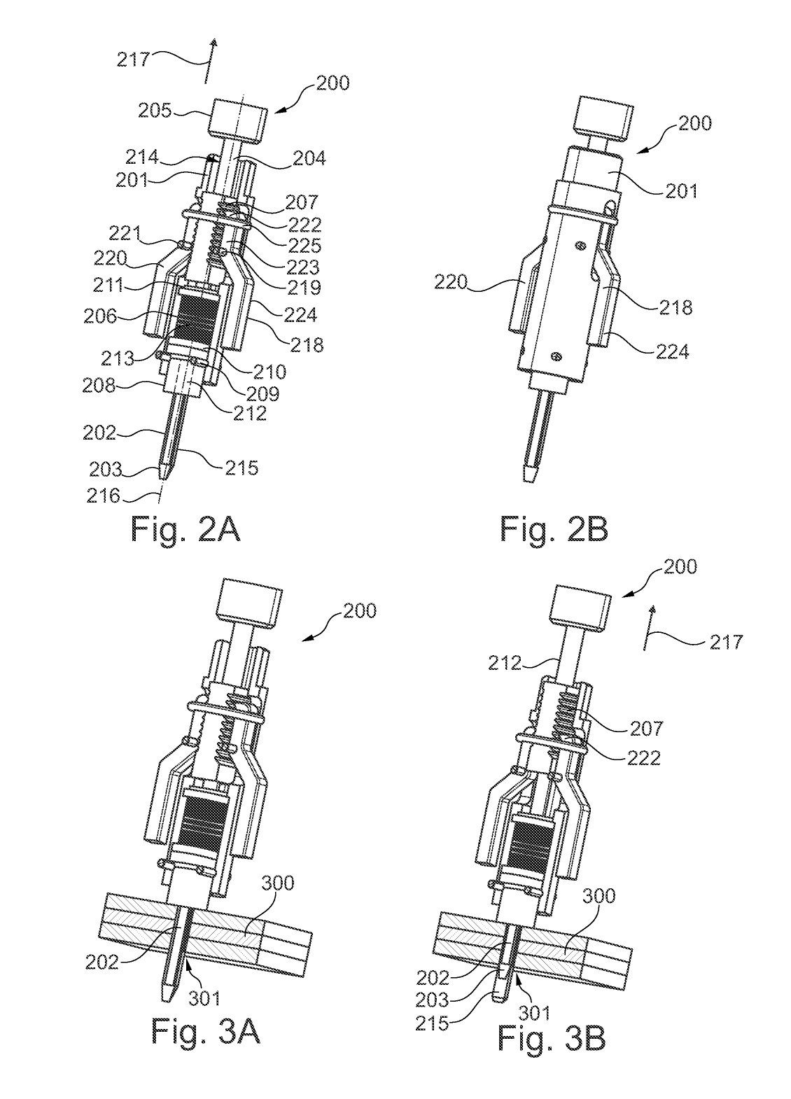

FIG. 2A presents an open sectional view of a stapling device 200 according to an exemplary embodiment of the invention.

The stapling device 200 exhibits a base body 201 and a stapling needle system 212. Situated in the base body 201 is an interior space 214, in which the stapling needle system 212 is guided. The interior space 214 is configured in such a way that the stapling needle system 212 can only be displaced in the direction of the longitudinal axis 216 of the stapling needle system 212.

The stapling needle system 212 encompasses a stapling needle 202, which exhibits a stapling needle head 203, as well as a stapling needle axis 204. The stapling needle 202 is rigidly joined with the stapling needle axis 204. This means that a movement along the longitudinal axis 216 of the stapling needle axis 204 is also accompanied by a movement along the longitudinal axis 216 of the stapling needle 202. A threaded ring 205 is situated at the upper end of the stapling needle axis. Screwing the threaded ring 205 makes it possible to adjust the distance between the base body 201 and threaded ring 205. For example, the distance between the base body 201 and threaded ring 205 can be adjusted to the size of the stapling tongs.

The stapling device 200 also encompasses a flexible force transmission element 206, which in the depicted exemplary embodiment of the invention is designed as a plate spring packet. This force transmission element 206 is situated in a second interior space 213 of the base body, wherein the stapling needle 202 is passed through the middle of the force transmission element, as denoted by the dashed lines. This means that there is an opening and/or a hole in the middle of the force transmission element through which the stapling needle 202 is passed. In addition, two disks 210 and 211 are situated at the two ends of the force transmission element 206. A thrust bushing 208 is also situated at the end of the force transmission element 206 facing the stapling needle head 203. This thrust bushing 208 is held in the interior space 213 of the base body by means of a cylindrical pin 209, which is guided through the interior space 213 of the base body 201 laterally to the longitudinal axis 216 of the stapling needle system 212 and perpendicular to the stapling needle system 212. However, there are other ways that the thrust bushing 208 can be held in the interior space 213 of the base body 201. The thrust bushing 208 also exhibits a hole, through which the stapling needle 202 is guided and can be displaced. In addition, the stapling device 200 encompasses a stapling needle tongue 215, which is guided in a groove of the stapling needle 202. The stapling needle 202 can also exhibit two halves which each have a stapling needle head half, wherein the stapling needle tongue is situated between the two halves. Both stapling needle head halves are forced apart when retracting the stapling needle system, thereby generating the clamp.

The stapling device 200 further comprises a locking device, which is constructed as follows. Situated on the stapling needle axis 204 is a tooth system 207, which is configured as a single piece with the stapling needle axis 204. Two locking levers 218 and 220 are pivoted to the base body 201 on a respective fulcrum 219 and 221 of the respective locking lever 218 and 220. The locking lever 218 contains a first arm 223, on which is situated a tooth element 222 pointing inwardly or in the direction of the tooth system 207 situated on the stapling needle axis. The tooth element 222 and locking lever 218 are designed as a single piece, so that the tooth element 222 projects inwardly out of the first arm 223 of the locking lever 218. The locking lever 218 also exhibits a second arm 224, which is situated outside of the base body 201, and can be actuated for undoing the positive connection between the tooth element 222 and tooth system 207. The same also holds true for the second locking lever 220. A force element or tension spring or a hose spring washer 225 further encompasses the respective first arm 222 of the locking levers 218 and 220, as well as the base body 201, thereby exerting a force on the tooth element 222 in the direction of the tooth system 207, so as to automatically lock the stapling device 200.

By exerting a tensile force 217 on the stapling needle axis 204, with the tensile force being exerted in a direction away from the stapling needle 202 and/or the component situated between the stapling needle head 203 and thrust bushing 208, the entire stapling needle system is displaced along the direction of the tensile force 217. This means that exerting the tensile force 217 retracts the stapling needle 202 relative to the base body 201. The distance between the thrust bushing 208 and stapling needle head 203 is decreased in this way. Retracting the stapling needle 202 guides the stapling needle tongue 215 through the groove of the stapling needle head 203. In this way, the stapling needle head is spread apart, or the diameter of the stapling needle head 203 is enlarged. Therefore, when the stapling needle 202 is guided through a hole in one or more components, and the stapling needle system 212 is retracted, the stapling needle head 203 spreads apart, so that it no longer fits through the opening of the component or components. In this way, components can be sandwiched between the stapling needle head 203 and thrust bushing 208. After the component or components have been sandwiched between the stapling needle head 203 and thrust bushing 208, if a tensile force 217 continues to be exerted on the stapling needle system 212, this tensile force 217 can be absorbed by the flexible force transmission element 206. This means that, if a clamping force continues to be applied to the component or components, this clamping force is transmitted to the flexible force transmission element 206 via the thrust bushing 208. The maximum clamping force exerted on the component or components can be limited in this way.

When retracting the stapling needle system 201, the tooth element 222 automatically latches or engages into the tooth system 207 via the force element 225, so that the stapling device is automatically locked in place, and the achieved clamp no longer releases by itself. In order to again undo the positive fit between the tooth element 222 and tooth system 207 and release the clamp once more, the two locking levers 220 and 224 can be pressed together with a simple manual operation.

FIG. 2B shows a closed stapling device 200 according to an exemplary embodiment of the invention, wherein only selected reference numbers are indicated. The base body encompasses the tooth mechanism depicted on FIG. 2A as well as the flexible force element 206. The second arm 224 of the locking lever 218 is here situated outside the base body 201, and can be actuated to release the stapling device 200. The same also holds true for the locking lever 220.

FIG. 3A shows how the stapling needle 202 of the stapling device 200 according to an exemplary embodiment of the invention is passed through holes 201 in several components 300 for clamping the components 300. FIG. 3B shows how the stapling needle system 212 is retracted by exerting a force 217. In this way, the stapling needle head 203 is pulled over the stapling needle tongue 215 and spread apart, so that the stapling needle head no longer fits through the holes 301, and the components are clamped. At the same time, the tooth element 222 latches into the tooth system 207 of the stapling needle axis, so as to prevent the stapling device 200 from detaching on its own.

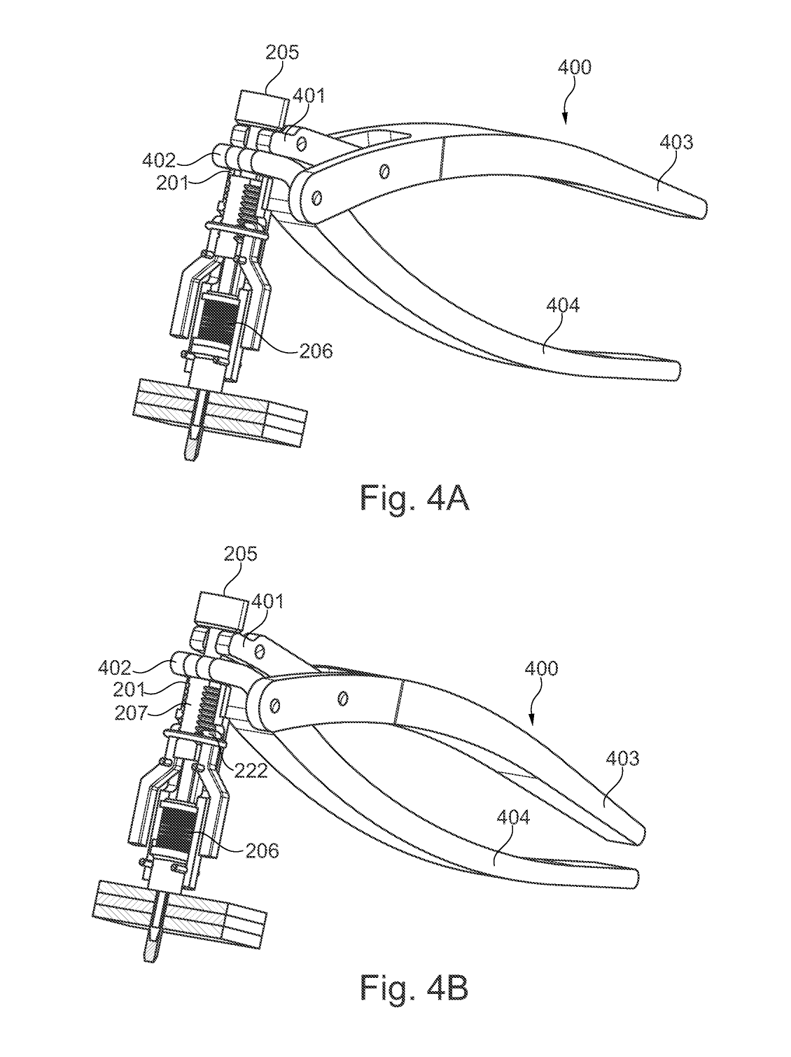

FIGS. 4A and 4B show how a larger clamping force is generated with a stapling device according to an exemplary embodiment of the invention. After the stapling needle system has been manually retracted as described above, stapling tongs 400 are secured between the base body 201 and threaded ring 205 according to FIG. 4A. The stapling tongs 400 exhibit two expansion jaws 401 and 402, which are spread apart by pressing together the levers 403 and 404 of the stapling tongs 400. This makes it possible to generate a larger clamping force, which can be limited by the force transmission element 206, as described in the context of the invention. FIG. 4B shows how the two levers 403 and 404 of the stapling tongs 400 are pressed together, so that the expansion jaws 401 and 402 are spread apart. In this way, the stapling needle system is moved further upward relative to the base body 201, and a larger clamping force of the components is generated between the thrust bushing and stapling needle head. If this clamping force exceeds a limit, the force transmission element 206 yields, thereby effectively limiting the clamping force. The force transmission element 206 can also be used to balance out the discrete distance between the teeth of the tooth system 207, so as to ensure a uniform clamping force given different component thicknesses.

For example, the stapling system described in the context of the invention encompasses the depicted stapling device 200 as well as the stapling tongs 400.

In addition, let it be noted that “encompassing” does not preclude any other elements or steps, and that “a” or “an” do not rule out a plurality. Let it further be noted that features or steps described with reference to one of the above exemplary embodiments can also be used in combination with other features or steps in other exemplary embodiments described above. Reference numbers in the claims are not to be construed as a limitation.

While at least one exemplary embodiment of the present invention(s) is disclosed herein, it should be understood that modifications, substitutions and alternatives may be apparent to one of ordinary skill in the art and can be made without departing from the scope of this disclosure. This disclosure is intended to cover any adaptations or variations of the exemplary embodiment(s). In addition, in this disclosure, the terms “comprise” or “comprising” do not exclude other elements or steps, the terms “a” or “one” do not exclude a plural number, and the term “or” means either or both. Furthermore, characteristics or steps which have been described may also be used in combination with other characteristics or steps and in any order unless the disclosure or context suggests otherwise. This disclosure hereby incorporates by reference the complete disclosure of any patent or application from which it claims benefit or priority.

1. A stapling device for temporarily clamping a component, the stapling device comprising:
a stapling needle system comprising a stapling needle and stapling needle head;
a base body having an interior space for guiding the stapling needle system; and
a locking device for locking the stapling needle system relative to the base body with a positive fit;
wherein the stapling needle system is configured to be displaced relative to the base body by applying a tensile force to the stapling needle system in the direction of a longitudinal axis of the stapling needle system in the interior space of the base body; and
wherein the stapling device is configured to clamp the component between the stapling needle head and base body.

2. The stapling device of claim 1, wherein the locking device comprises:
a tooth system situated on the base body;
a tooth element situated on the base body; and
wherein the tooth element situated on the base body for positively locking the stapling needle system is configured to engage into the tooth system situated on the stapling needle system.

3. The stapling device of claim 2, wherein the locking device further comprises:
a first locking lever pivoted to the base body;
wherein the first locking lever comprises the tooth element; and
wherein the stapling device comprises a force element for exerting a force on the first locking lever, the force element configured to press the tooth element in the direction of the tooth system situated on the stapling needle system.

4. The stapling device of claim 3, wherein the locking lever comprises a first arm, a second arm and a fulcrum situated between the first arm and second arm;
wherein the fulcrum pivotably attaches the locking lever to the base body;
wherein the tooth element is situated on the first arm; and
wherein the second arm is configured to be actuated to release the stapling needle system.

5. The stapling device of claim 4, wherein the force element is a tension spring situated around the first arm and around the base body, or a hose spring washer.

6. The stapling device of claim 3, wherein the locking device further comprises a second locking lever.

7. The stapling device of claim 1, the stapling device further comprising:
a flexible force transmission element for transmitting a clamping force to the component;
wherein the force transmission element is secured to the base body in such a way that, when the components are being sandwiched between the base body and stapling needle head, the force transmission element is situated between the base body and component, so that the clamping force can be transmitted from the base body to the force transmission element, and from the force transmission element to the components.

8. The stapling device of claim 7, wherein the maximum clamping force that can be transmitted to the component is limited by having the force transmission element transmit the clamping force to the component.

9. The stapling device of claim 7, wherein the flexible force transmission element is a spring element.

10. The stapling device of claim 7, wherein the stapling device further comprises:
a thrust bushing configured to exert a clamping force on the component;
wherein the force transmission element is situated in the interior space of the base body; and
wherein the thrust bushing is situated on the force transmission element in such a way that, while clamping the components, the clamping force can be transmitted from the force transmission element to the thrust bushing, and from the thrust bushing to the components.

11. A stapling system, comprising:
a stapling device comprising:
a stapling needle system comprising a stapling needle and stapling needle head;
a base body having an interior space for guiding the stapling needle system; and
a locking device for locking the stapling needle system relative to the base body with a positive fit;
wherein the stapling needle system is configured to be displaced relative to the base body by applying a tensile force to the stapling needle system in the direction of a longitudinal axis of the stapling needle system in the interior space of the base body; and
wherein the stapling device is configured to clamp the component between the stapling needle head and base body, and
stapling tongs comprising first and second expansion jaws configured to transmit the clamping force to the base body and stapling needle system, thereby leading to a displacement of the stapling needle system relative to the base body.
